(19) United States
(12) Patent Application Publication
(10) Pub. No.: US 2026/0248883 A1
(43) Pub. Date: Aug. 27, 2026

(54) Composition for preventing or treating brain disorders

(71) Applicant: RESEARCH & BUSINESS FOUNDATION SUNGKYUNKWAN UNIVERSITY, Suwon-si (KR)

(72) Inventors: JEE YIN AHN, Seoul (KR); Byeong-Seong Kim, Gwacheon-si (KR)

(21) Appl. No.: 19/298,385

(22) Filed: Aug. 13, 2025

(30) Foreign Application Priority Data
Aug. 14, 2024 (KR) 10-2024-0109215

(51) Int. Cl. A61K 38/17; A61K 48/00; A61P 25/28

(52) U.S. Cl. CPC A61K 38/1709; A61K 48/0033; A61K 48/005; A61P 25/28

(57) ABSTRACT

Disclosed is a pharmaceutical composition for preventing or treating a brain disease, containing as an active ingredient an ErbB3-binding protein 1 (EBP1) protein or a polynucleotide sequence encoding the EBP1 protein, wherein an EBP1 N84A/N204A protein, which is an EBP1 mutant in an asparagine endopeptidase (AEP)-uncleavable form, and a polynucleotide encoding the same can reduce amyloid beta production and enhance cognitive function in the early stages of Alzheimer's disease, and thus the composition can be advantageously used as a therapeutic agent for brain diseases including sporadic Alzheimer's disease.

### REFERENCE TO AN ELECTRONIC SEQUENCE LISTING

The content of the electronic sequence listing (ZPN20250017US_SEQ.xml; Size: 13,907 bytes; and Date of Creation: Aug. 13, 2025) is herein incorporated by reference in its entirety. The contents of the electronic sequence listing in no way introduces new matter into the specification.

### BACKGROUND OF THE INVENTION

### 1. Field of the Invention

The present disclosure was made with the support of the multiple government ministries in the Republic of Korea, under Project ID 1465039785 and Subproject No. HU21C0157000023, which was conducted in the research project named “Identification of neural degenerative mechanism by Ebp1 dysfunction for the new etiological mechanisms and treatment targets for Alzheimer's disease dementia” in the research program titled “Research and Development Program for Overcoming Dementia (Ministry of Health Welfare and Ministry of Science and ICT)”, by Sungkyunkwan University, under the management of the Korea Health Industry Development Institute, from 1 Jan. 2023 to 31 Dec. 2023.

This application claims priority and the benefit of Korean Patent Application No. 10-2024-0109215 filed in the Korean Intellectual Property Office on 14 Aug. 2024, the disclosure of which is incorporated herein by reference.

The present disclosure relates to a pharmaceutical composition for preventing or treating Alzheimer's disease, containing as an active ingredient an EBP1 protein or a nucleic acid including a polynucleotide sequence encoding the EBP1 protein.

### 2. Description of the Prior Art

Alzheimer's disease is a neurodegenerative disease characterized by gradual neuronal death and cognitive impairment due to the abnormal accumulation of amyloid beta or hyperphosphorylated tau proteins. The advent of an aging society results in an increased incidence of Alzheimer's disease, and thus active research and development for therapeutics therefor is underway (as of January 2023, “The Common Alzheimer's Disease Research Ontology (CADRO)” reported 187 Phase 1, 2, and 3 clinical trials for 141 drugs), but effective FDA-approved therapeutics are still lacking.

The targets for developing Alzheimer's disease therapeutics mainly involve regulating neurotransmitter receptors and synaptic plasticity and inhibiting immune responses, and also include mechanisms of direct removal of amyloid beta or tau protein accumulations. Lecanemab and Aducanumab, currently approved as treatments for Alzheimer's disease by FDA, have been revealed to be effective in removing amyloid beta. However, the substances possess properties of binding well to oligomeric or fibrillar forms of amyloid beta with already increased sizes, rather than monomeric forms, and have a limitation that they can remove only already generated amyloid beta.

Therefore, it is urgent to develop therapeutics that can treat Alzheimer's disease in its early stages by inhibiting amyloid beta production.

### PRIOR ART DOCUMENT

### Patent Document

(Patent Document 01) Korean Patent No. 10-2526556 (2 May 2023)

### SUMMARY OF THE INVENTION

The present inventors have made intensive efforts to develop a therapeutic that can treat Alzheimer's disease in its early stages by inhibiting amyloid beta production. As a result, the present inventors established that an EBP1 N84A/N204A protein, which is an EBP1 mutant in an asparagine endopeptidase (AEP)-uncleavable form, and a polynucleotide encoding the same can reduce amyloid beta production and enhance cognitive function in the early stages of Alzheimer's disease, and thus the present disclosure has been completed.

Accordingly, an aspect of the present disclosure is to provide an ErbB3-binding protein 1 (EBP1) protein including a sequence set forth in any one of SEQ ID NOs: 1 to 3.

Another aspect of the present disclosure is to provide a nucleic acid molecule including a polynucleotide sequence encoding the EBP1 protein.

Still another aspect of the present disclosure is to provide a pharmaceutical composition for preventing or treating brain diseases, containing as an active ingredient an EBP1 protein or a nucleic acid including a polynucleotide sequence encoding the EBP1 protein.

The present inventors have made intensive efforts to develop a therapeutic that can treat Alzheimer's disease in its early stages by inhibiting amyloid beta production. As a result, the present inventors established that an EBP1 N84A/N204A protein, which is a mutant having an asparagine endopeptidase (AEP)-uncleavable form, and a polynucleotide encoding the same reduced amyloid beta production and enhanced cognitive function in the early stages of Alzheimer's disease.

The present disclosure is directed to a pharmaceutical composition for preventing or treating Alzheimer's disease, containing as an active ingredient an EBP1 protein or a nucleic acid including a polynucleotide sequence encoding the EBP1 protein.

Hereinafter, the present disclosure will be described in more detail.

In accordance with an aspect of the present disclosure, an ErbB3-binding protein 1 (EBP1) protein including a sequence set forth in any one of SEQ ID NOs: 1 to 3 is provided.

As used herein, the term “ErbB3-binding protein 1 (EBP1)” refers to a gene that is located at chromosome band 12q13.2, over-expressed in various cancer cells, such as brain tumors and breast cancer, and is strongly expressed during brain development, rapidly reduced after birth, and then gradually reduced during in adults. Starting with global histone methylation in 2019, the involvement of Ebp1 in epigenetics has been consistently reported.

The EBP1 protein of the present disclosure has a mutation at asparagine 84 or 204 of an EBP1 protein.

A peptide containing the mutated sequence at position 84 of the EBP1 protein is set forth in SEQ ID NO: 1, hereinafter referred to as N84A. A peptide containing the mutated sequence at position 204 of the EBP1 protein is set forth in SEQ ID NO: 2, hereinafter referred to as N204A. A peptide containing the mutated sequences at positions 84 and 204 of the EBP1 protein is set forth in SEQ ID NO: 3, hereinafter referred to as N84A/N204A.

In accordance with another aspect of the present disclosure, a nucleic acid including a polynucleotide sequence encoding the EBP1 protein is provided.

In an embodiment of the present disclosure, the polynucleotide encoding the EBP1 protein induces overexpression of the EBP1 protein in a host cell transfected with the polynucleotide. The host cell is, for example, a neuron, and the neuron cell may be derived from the central nervous system, specifically the brain or spinal cord, and more specifically the cerebrum, forebrain, midbrain, cerebellum, diencephalon, or spinal cord, but is not limited thereto.

In an embodiment of the present disclosure, the polynucleotide encoding the EBP1 protein induces transient or stable overexpression of the EBP1 protein in the transfected host cell.

As used herein, the term “overexpression” refers to an increased expression of a gene of interest. The term overexpression includes a comprehensive meaning that encompasses increasing transcription and/or translation of a gene of interest by a method of cloning the gene of interest into an expression vector and transfecting the gene into cells, or by other methods.

In accordance with still another aspect of the present disclosure, a pharmaceutical composition for preventing or treating brain diseases, containing as an active ingredient an EBP1 protein or a nucleic acid including a polynucleotide sequence encoding the EBP1 protein is provided.

In an embodiment of the present disclosure, the EBP1 protein includes a sequence set forth in any one of SEQ ID NOs: 1 to 3.

In an embodiment of the present disclosure, the polynucleotide sequence includes a polynucleotide sequence set forth in any one of SEQ ID NOs: 4 to 6.

In an embodiment of the present disclosure, the polynucleotide is a naked DNA or is contained in a gene delivery carrier.

As used herein, the term “naked DNA” refers to DNA that is not associated with proteins, lipids, or other molecules that help protect DNA.

As used herein, the term “gene delivery carrier” refers to a DNA molecule that serves to deliver an exogenous DNA fragment, which is inserted into its own DNA.

In an embodiment of the present disclosure, the gene delivery carrier is a vector.

As used herein, the term “vector” refers to any means for expressing a target gene in a host cell, and examples thereof include phagemid vectors, plasmid vectors, cosmid vectors, bacteriophage vectors, and viral vectors, such as adenoviral vectors, retroviral vectors, and adeno-associated viral vectors, but are not limited thereto. Since plasmids are currently one of the most commonly used forms of vectors, the terms “plasmid” and “vector” may be used interchangeably in the present disclosure.

In an embodiment of the present disclosure, the vector may be a recombinant vector.

In an embodiment of the present disclosure, the plasmid may be a recombinant plasmid.

According to an embodiment of the present disclosure, the polynucleotide encoding EBP1 protein is operatively linked to a promoter in the vector of the present disclosure.

As used herein, the wording “operatively linked” refers to a functional linkage between a nucleic acid expression control sequence (e.g., a promoter, a signal sequence, or an array of transcription factor binding sites) and another nucleic acid sequence, whereby the control sequence directs the transcription and/or translation of the other nucleic acid sequence.

The vector or recombinant vector of the present disclosure may be constructed by various methods known in the art, and specific methods therefor are disclosed in Sambrook et al., Molecular Cloning, A Laboratory Manual, Cold Spring Harbor Laboratory Press (2001), which is incorporated herein by reference.

The vector of the present invention may be typically constructed as a vector for cloning or a vector for expression. The vector for cloning of the present disclosure may be constructed by using a prokaryotic or eukaryotic cell as a host. Additionally, the vector for cloning of the present disclosure contains a multiple cloning site (MCS).

As used herein, the “promoter” is used to promote the expression of a gene to be transfected, and the promoter may further include not only a basal element necessary for transcription, but also an enhancer that may be used to promote and regulate the expression.

For example, in cases where the vector of the present disclosure is an expression vector and an eukaryotic cell is used as a host cell, promoters derived from the genomes of mammalian cells (e.g., metallothionein promoter, beta-actin promoter, human hemoglobin promoter, and human muscle creatine promoter) or promoters derived from mammalian viruses (e.g., adenovirus late promoter, vaccinia virus 7.5 K promoter, SV40 promoter, cytomegalovirus promoter, HSV tk promoter, mouse mammary tumor virus (MMTV) promoter, HIV LTR promoter, Moloney virus promoter, Epstein-Barr virus (EBV) promoter, and Rous sarcoma virus (RSV) promoter) may be used, and it typically comprises a polyadenylation sequence as a transcription termination sequence.

In an embodiment of the present disclosure, the promoter may be an elongation factor 1 alpha (EF1α) promoter or a cytomegalovirus (CMV) promoter, but is not limited thereto.

The vector of the present disclosure may be fused with another sequence in order to facilitate the purification of proteins expressed therefrom. Examples of the sequences for fusion include glutathione S-transferase (Pharmacia, USA), maltose-binding protein (NEB, USA), FLAG (IBI, USA), 6×His (hexahistidine, Quiagen, USA), and the like.

The vector of the present disclosure includes, as a selective marker, an antibiotic-resistant gene that is commonly used in the art, and examples thereof include antibiotics resistance genes for ampicillin, gentamicin, carbenicillin, chloramphenicol, streptomycin, kanamycin, geneticin, neomycin, and tetracycline.

Optionally, the vector may additionally deliver a gene encoding a reporter molecule (e.g., luciferase and -glucuronidase).

As used herein, the term “brain disease” refers to Alzheimer's disease, but is not limited thereto.

As used herein, the term “Alzheimer's disease (AD)” refers to an irreversible, progressive brain disease that causes cognitive and functional impairment, affecting memory, thinking ability, spatial orientation, and personality, and in the most severe cases, may even affect the ability to perform the most basic activities of daily living. Alzheimer's disease is a disease characterized by the accumulation of amyloid beta arising from complex neurodegenerative mechanisms associated with age-associated genetic mutations or brain injury progression.

The present inventors identified i) an increase in gamma-secretase activity and the accumulation of amyloid beta, and ii) behavioral changes, such as reduced sociability and cognitive function in the Morris water maze test, passive avoidance test, Y-maze test, and novel object recognition test in 5X-FAD mice representatively known as an Alzheimer's disease animal model. Moreover, the administration of EBP1 N84A/N204A protein-overexpression vectors to the 5X-FAD mice resulted in the recovery of the above-described morphological and behavioral changes.

Therefore, the above results suggested that the pharmaceutical composition of the present disclosure containing as an active ingredient an EBP1 protein or a polynucleotide encoding the EBP1 protein can prevent or treat a brain disease in a subject (or patient) with down-regulated EBP1 gene. The subject (or patient) is characterized by a subject (patient) having Alzheimer's disease.

That is, the subject or patient group to be administered the composition of the present disclosure is a brain disease patient group with down-regulated EBP1 gene compared to a normal control group, characterized by having Alzheimer's disease.

As used herein, the term “down-regulation” refers to a lower level of expression of a gene or a protein encoded by the gene as compared to a normal control. As used herein, the term “up-regulation” refers to a higher level of expression of a gene or a protein encoded by the gene as compared to a normal control

As used herein, the term “prevention” refers to any action that inhibits or delays the onset of the brain disease in a subject by the administration of the composition according to the present disclosure, and the term “treatment” refers to any action that alleviates the brain disease or advantageously change the disease status in a subject by the administration of the composition.

The pharmaceutical composition of the present disclosure may be applied to not only humans, but also mammals that may develop the brain disease, such as cattle, horses, sheep, pigs, goats, camels, antelopes, dogs, and cats.

The pharmaceutical composition of the present disclosure may further contain an appropriate carrier, excipient, or diluent according to a commonly used method.

Examples of a carrier, an excipient, and a diluent that may be contained in the composition of the present disclosure may include lactose, dextrose, sucrose, sorbitol, mannitol, xylitol, erythritol, maltitol, starch, acacia rubber, alginate, gelatin, calcium phosphate, calcium silicate, cellulose, methyl cellulose, microcrystalline cellulose, polyvinyl pyrrolidone, water, methyl hydroxybenzoate, propyl hydroxybenzoate, talc, magnesium stearate, and a mineral oil.

The composition of the present disclosure may be formulated in an oral dosage form, such as a powder, granules, a tablet, a capsule, a suspension, an emulsion, a syrup, or an aerosol, or in the form of an external preparation, a suppository, and a sterile injectable solution, according to a commonly used method for each form.

Specifically, the composition may be formulated into preparations by using a diluent orexcipient, such as a filler, extender, binder, humectant, disintegrant, or surfactant. Solid preparations for oral administration include tablets, pills, powders, granules, capsules, and the like, and such solid preparations may be prepared by mixing the active ingredient with at least one excipient, for example, starch, calcium carbonate, sucrose, lactose, or gelatin. In addition, lubricants, such as magnesium stearate and talc, may be used in addition to simple excipients. Liquid preparations for oral administration include suspensions, solutions, emulsions, and syrups, and may contain various excipients, such as wetting agents, sweeteners, aromatics, and preservatives, in addition to commonly used simple diluents, such as water and liquid paraffin. Preparations for parenteral administration include a sterile aqueous solution, a non-aqueous solvent, a suspending agent, an emulsion, a freeze-dried preparation, and a suppository. Examples of the non-aqueous solvent and suspending agent may include propylene glycol, polyethylene glycol, a vegetable oil such as olive oil, an injectable ester such as ethyl oleate, and the like. Examples of a base for the suppository may include Witepsol, Macrogol, Tween 61, cocoa butter, laurin butter, glycerogelatin, and the like.

The preferred dosage of the active ingredient of the present disclosure may vary depending on the patient's condition, age, and body weight, the severity of the disease, drug form, and route and period of administration, but may be selected as appropriate by a person skilled in the art. However, for desirable efficacy, the active ingredient of the present disclosure may be administered at 0.0001 to 100 mg/kg, and preferably, 0.001 to 100 mg/kg, in a single dose or divided into multiple doses per day. In the composition, the active ingredient of the present disclosure needs to be present in an amount of 0.0001 to 10 wt %, and preferably 0.001 to 1 wt % relative to the total weight of the composition.

The pharmaceutical dosage form of the active ingredient of the present disclosure may also be used in the form of a pharmaceutically acceptable salt thereof, and may also be used alone or in combination or in an appropriate set with other pharmaceutically active ingredients.

The pharmaceutical composition of the present disclosure may be administered to mammals, such as mice, rats, cattle, horses, and pigs, through various routes. All modes of administration may be contemplated, and for example, the administration may be performed via oral, intraperitoneal, rectal, or inhalation routes or by intravenous, arterial, intramuscular, transdermal, subcutaneous, intradermal, intrauterine, intracerebral, intracerebroventricular, intrathecal, or intracerebrovascular injection.

The present disclosure is directed to a pharmaceutical composition for preventing or treating a brain disease, containing as an active ingredient an EBP1 protein or a nucleic acid including a polynucleotide sequence encoding the EBP1 protein, and the use of the composition of the present disclosure can reduce the production of amyloid beta in an initial stage of Alzheimer's disease by EBP1 N84A/N204A protein, which is an EBP1 mutant in an asparagine endopeptidase (AEP)-uncleavable form, and a polynucleotide encoding the mutant, and thus can be advantageously used as a therapeutic agent for early-stage Alzheimer's disease.

### BRIEF DESCRIPTION OF THE DRAWINGS

The above and other aspects, features and advantages of the present disclosure will be more apparent from the following detailed description taken in conjunction with the accompanying drawings.

FIG. 1 illustrates reduced EBP1 expression in the brains of Alzheimer's disease patients.

FIG. 1A shows the results of analyzing EBP1 RNA expression.

FIG. 1B shows the result of analyzing the relationship between EBP1 protein expression and amyloid beta accumulation by immunohistochemistry.

FIG. 1C shows the results of analyzing the relationship between EBP1 protein expression and the number of neurons.

FIG. 1D shows the result of analyzing the relationship between EBP1 protein expression and amyloid beta accumulation by Western blotting.

FIG. 2 illustrates that EBP1 is a substrate of AEP and AEP-mediated EBP1 cleavage occurs in Alzheimer's disease.

FIG. 2A shows the results of analyzing EBP1 cleavage in Alzheimer's disease by Western blotting.

FIG. 2B shows the results of confirming the binding between EBP1 and AEP in cells by Western blotting.

FIG. 2C shows the results of confirming the production of two fragments by AEP-mediated EBP1 cleavage in cells.

FIG. 2D shows the results of confirming the abolishment of EBP1 cleavage by the treatment with AENK, an AEP peptide inhibitor.

FIG. 2E shows the results of confirming the site of AEP action on EBP1 through mutagenesis experiments.

FIG. 2F shows the results of confirming the site of AEP-mediated EBP1 cleavage through mass spectrometry.

FIG. 3 illustrates that the EBP1 fragments EBP1 1-84 and EBP1 1-204 were specifically observed in the brain tissues of Alzheimer's disease patients.

FIG. 3A is a schematic diagram of the construction of EBP1 71-84 fragment- and EBP1 191-204 fragment-specific antibodies. The EBP1 71-84 and EBP1 191-204 antibodies can specifically detect EBP1 1-84 and EBP1 1-204 fragments, respectively.

FIG. 3B shows the results of detecting EBP1 fragments by the constructed EBP1 71-84 fragment- and EBP1 191-204 fragment-specific antibodies.

FIG. 3C shows the results of comparing the detections of EBP1 1-84 and EBP1 1-204 fragments in the brain tissues of controls and Alzheimer′ disease patients.

FIG. 3D shows the results of treatment with EEA1, LAMP1, and EBP1 71-84 fragment- and EBP1 191-204 fragment-specific antibodies in the brain tissues of controls and Alzheimer′ disease patients.

FIG. 3E shows the results of Western blotting of brain tissues at different month ages in the Alzheimer animal model 5X-FAD mice.

FIG. 4 confirms that EBP1 fragments induced neuronal toxicity.

FIG. 4A is a schematic diagram of experimentation for investigating cytotoxicity by EBP1 1-84 and 1-204.

FIG. 4B shows the TUNEL staining results of confirming an increase in amyloid beta production in primary neurons with apoptosis occurring by EBP1 fragments.

FIG. 4C shows the results of measuring the axon length of neurons following the overexpression of AEP-uncleavable EBP1.

FIG. 4D shows the results of confirming the accumulation of EBP1 1-84 and 1-204, and amyloid beta in the brain tissues of Alzheimer's disease patients.

FIG. 5 shows the results of confirming that EBP1 can regulate the cleavage of APP by gamma-secretase.

FIG. 5A shows the results of measuring gamma-secretase activity in the brains in 5X-FAD and CKO mice.

FIG. 5B shows the results of confirming that EBP1 1-84 and 1-204 fragments increased gamma-secretase activity.

FIG. 5C shows the results of analyzing the effect of EBP1 expression on gamma-secretase-mediated cleavage of C99.

FIG. 5D shows the results of comparing and analyzing the bindings between EBP1 and gamma-secretase substrates in normal mouse models and 5X-FAD, through Western blotting and PLA assay.

FIG. 5E shows the results of confirming that the action of EBP1 on gamma-secretase affected only the activity of gamma-secretase toward APP.

FIG. 5F shows that EBP1 was uncleavable by gamma-secretase.

FIG. 5G shows the results of confirming that the overexpression of EBP1-wild-type and EBP1 N84A/N204A reduced gamma-secretase activity.

FIG. 5H shows the comparison of the binding levels of PSEN with other substrates through immunoprecipitation following the treatment of primary neurons with amyloid beta and overexpression of Flag-EBP1 at different concentrations.

FIG. 6 shows the results of confirming that the induction of EBP1-wild-type and EBP1 N84A/N204A protein expression in Alzheimer's disease animal models suppressed amyloid beta production, as well as promoted amyloid beta removal.

FIG. 6A is a schematic diagram of behavior tests and immunohistochemistry analysis after the injection of AAV-EBP1 and AAV-N84A/204A into the hippocampus of 5X-FAD mice.

FIG. 6B shows the Morris water maze test results.

FIG. 6C shows the passive evidence test results.

FIG. 6D shows the Y-maze test results.

FIG. 6E shows the novel object recognition test results.

FIG. 6F shows the results of confirming the reduction of amyloid beta accumulations after overexpression of EBP1 and N84A/N204A proteins.

FIG. 6G shows the results of confirming that the reduction of amyloid beta accumulations attributed to the reduction in binding between the APP and PSEN in cases of the overexpression of EBP1 and N84A/N204A.

FIG. 7 is a schematic diagram illustrating a therapeutic target mechanism of the composition for preventing or treating Alzheimer's disease of the present disclosure, that is, the AEP-mediated EBP1 cleavage causes neurodegeneration, leading to the deregulation of gamma-secretase and accelerating amyloid beta production.

### DETAILED DESCRIPTION OF THE EXEMPLARY EMBODIMENTS

Hereinafter, the present disclosure will be described in more detail with reference to exemplary embodiments. These exemplary embodiments are provided only for the purpose of specifically illustrating the present disclosure, and therefore, according to the purpose of the present disclosure, it would be apparent to a person skilled in the art that these exemplary embodiments are not construed to limit the scope of the present disclosure.

### EXAMPLES

Throughout the present specification, the “%” used to express the concentration of a specific material, unless otherwise particularly stated, refers to (wt/wt) % for solid/solid, (wt/vol) % for solid/liquid, and (vol/vol) % for liquid/liquid.

The data values presented herein are expressed as means±S.E.M. Statistical significance was calculated using Student's two-tailed unpaired t-test, multiple unpaired t-test, one-way ANOVA, and two-way ANOVA. Significance levels are indicated as * (p<0.05), ** (p<0.01), *** (p<0.001), ****(p<0.0001), and ns (not significant).

### Example 1: Analysis of Relationship Between EBP1 Protein and Alzheimer′ Disease

### 1-1. Analysis of EBP1 RNA Expression

The EBP1 RNA expression in the brains of an Alzheimer's disease patient group (AD), an age-matched non-Alzheimer's disease control group (Control), and a group ranging in age from 40 to under 106 years, was investigated using GEO datasets.

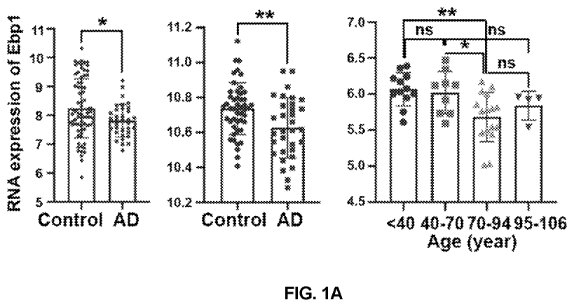

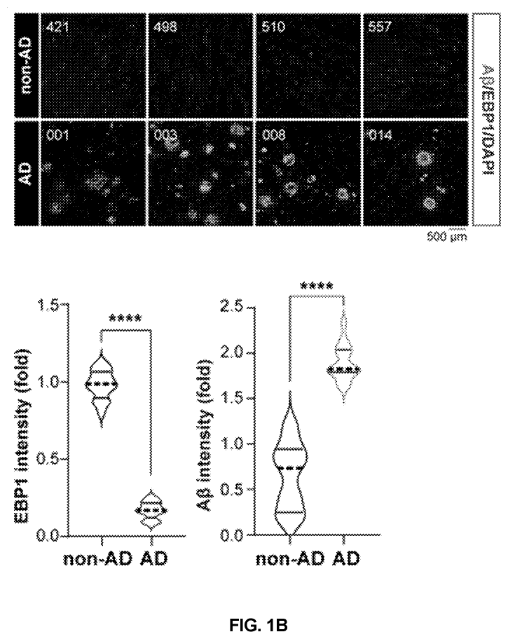

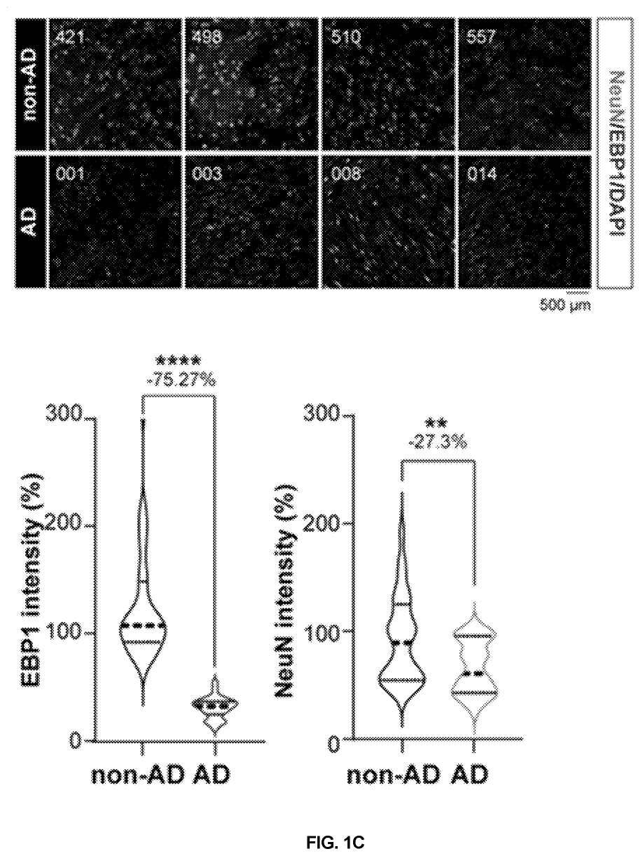

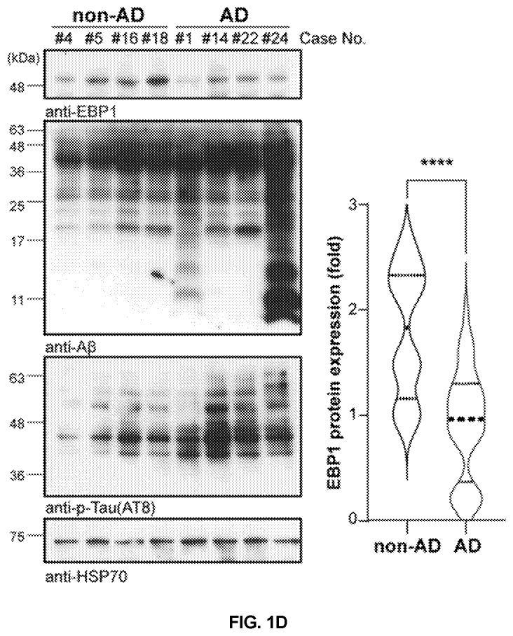

As confirmed by the results shown in FIG. 1A, the EBP1 RNA expression was significantly reduced in the brains of Alzheimer's disease patients and aged persons.

### 1-2. Analysis of EBP1 Protein Expression Using Immunostaining

The EBP1 expression changes in the postmortem brain tissues of Alzheimer's disease patients were analyzed. The postmortem brain tissues were obtained with approval of the Korea Centers for Disease Control and Prevention Agency (SMC 2016-11-032-063). The EBP1 expression changes were analyzed in the brain tissues from eight patients diagnosed with Alzheimer's disease and eight age-matched controls.

The brain tissues were post-fixed in 4% paraformaldehyde (PFA) and subsequently incubated with 30% sucrose. The tissues were cryosectioned, and the slices were permeabilized for 2 hours using 0.2% Triton X-100 in PBS, washed, and then blocked in PBS containing 2% BSA for 1 hour. Primary cultured neurons fixed with 4% PFA after lipofectamine transfection were blocked in 1% BSA and then permeabilized with 0.1% Triton X-100. The samples were immunostained overnight by using primary antibodies, following washing, and then incubated with secondary antibodies (Alexa Fluor-488 for green signal, -546 for red signal, and -405 for blue signal) for 2 hours at room temperature. DAPI was applied for nuclear counterstaining. The stained tissues and cells were mounted on slides using a mounting medium (Vector Laboratories, Burlingame, CA, USA; H-1400 or H-1500 with DAPI). Images were acquired as Z-stack images using a Zeiss LSM 710 confocal microscope. All images were analyzed with identical parameters by using ZEN and ImageJ software.

The results are shown in FIGS. 1B to 1D.

As confirmed by the immunostaining results shown in FIG. 1B, the EBP1 protein expression was rapidly reduced in the brain of Alzheimer's disease patients with extensive amyloid beta accumulation.

As confirmed by the immunostaining results shown in FIG. 1C, when the EBP1 protein expression was reduced, the number of neurons was also decreased.

As confirmed by the Western blotting results shown in FIG. 1D, the EBP1 protein expression was rapidly reduced in the brain of Alzheimer's disease patients with extensive amyloid beta accumulation.

### Example 2: Analysis of Interaction Between EBP1 Protein and Asparagine Endopeptidase (AEP)

By using brain tissues from Alzheimer's disease patients and 5X-FAD mice corresponding to Alzheimer's disease animal models, it was identified through Western blotting and mass spectrometry that EBP1 is a substrate of AEP and AEP-mediated cleavage of EBP1 occurred in Alzheimer's disease.

After protein cleavage assays were performed using purified EBP1 protein, EBP1 fragments visualized by Coomassie blue staining were analyzed by mass spectrometry. Chromatographic mass spectrometry analysis was performed using a nano ACQUITY UPLC coupled with an LTQ-orbitrap-mass spectrometer, and the corresponding analysis was commissioned to PROTIA.

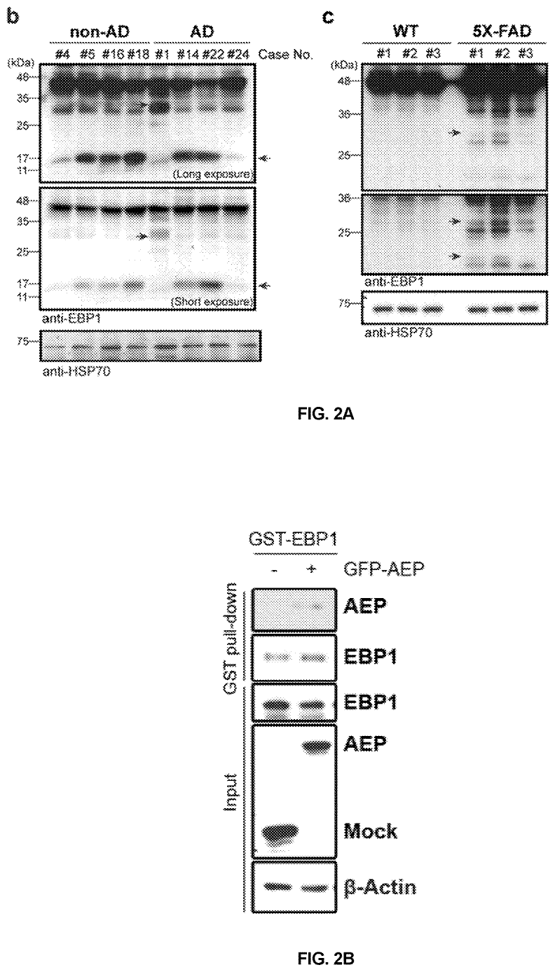

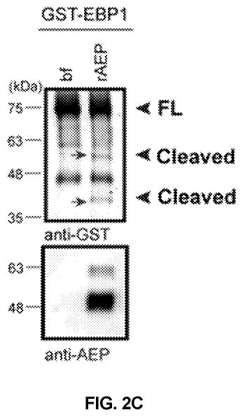

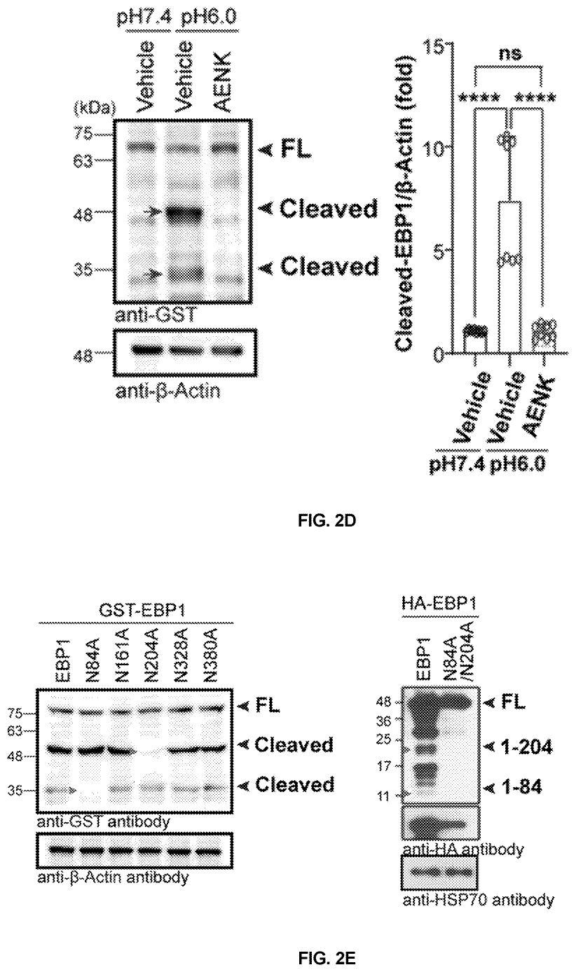

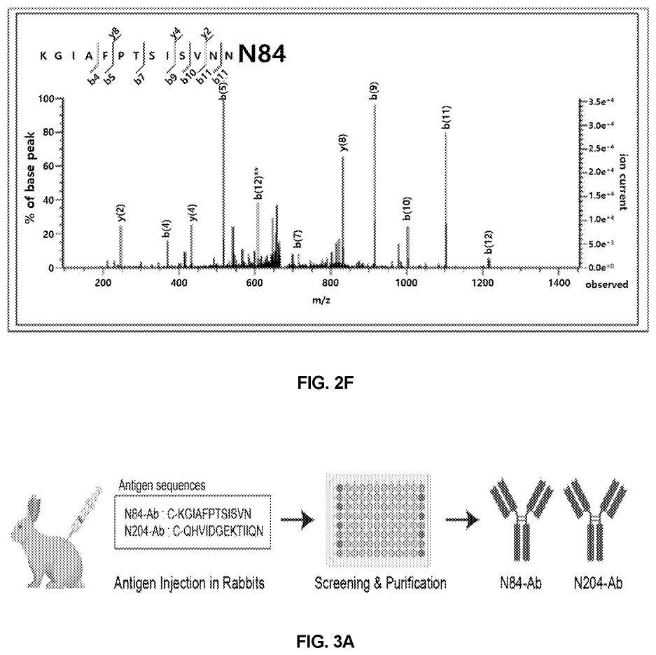

As confirmed by the Western blotting results shown in FIG. 2A, the EBP1 protein cleavage was observed in the brains of Alzheimer's disease patients (left) and 5X-FAD (right).

As confirmed by the Western blotting results shown in FIG. 2B, EBP1 and AEP could be bound to each other in the cells.

As confirmed by the in vitro EBP1 protein cleavage assay results shown in FIG. 2C, purified EBP1 was cleaved into two fragments by purified AEP protein activated at a low pH.

As shown in FIG. 2D, the cleavage of EBP1 was abolished by the AEP peptide inhibitor AENK.

As shown in FIG. 2E, as a result of protein cleavage assays on EBP1 protein with mutations at potential target asparagine (N) residues of AEP-cleavable sites, asparagine 84 and asparagine 204 were cleavable by AEP. It was also identified that the cleavage of EBP1 protein was abolished by introducing the mutations of asparagine 84 and asparagine 204 (N84A/N204A).

As shown in FIG. 2F, as a result of three independent experiments followed by analysis by mass spectrometry, EBP1 1-84 fragments as fragments cleaved at asparagine 84 of the EBP1 protein were more clearly obtained than EBP1 1-204 fragments as fragments cleaved at asparagine 204 of the EBP1 protein, indicating that the AEP-mediated EBP1 cleavage primarily occurred at asparagine 84.

### Example 3: Confirmation of EBP1 Fragments in Brain Tissues of Alzheimer's Disease Patients

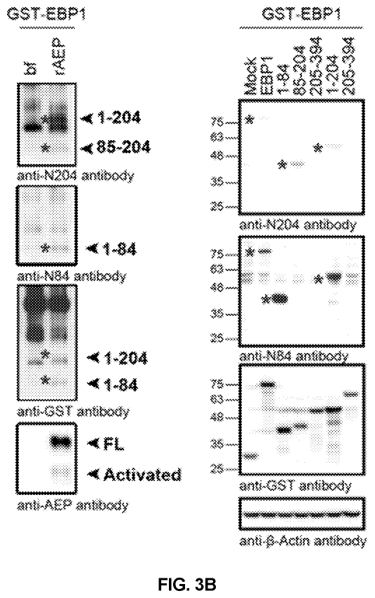

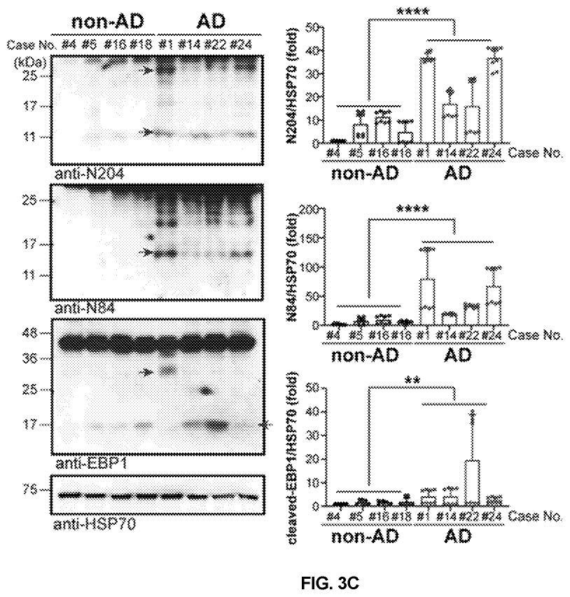

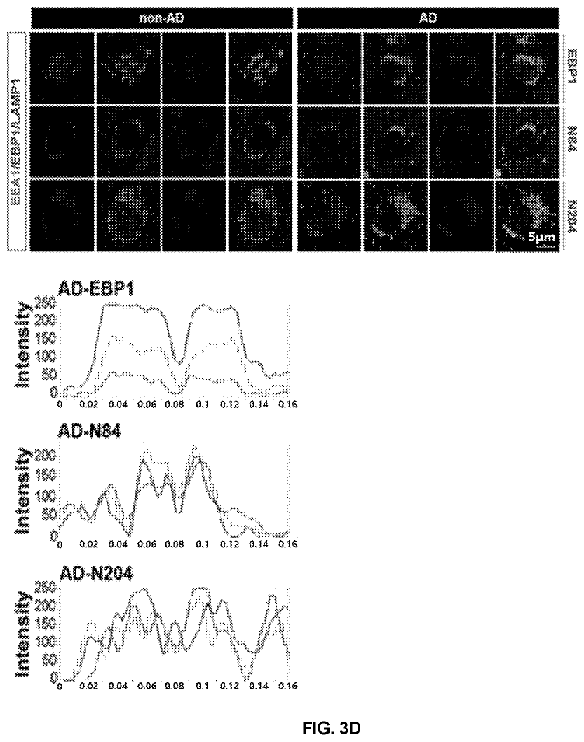

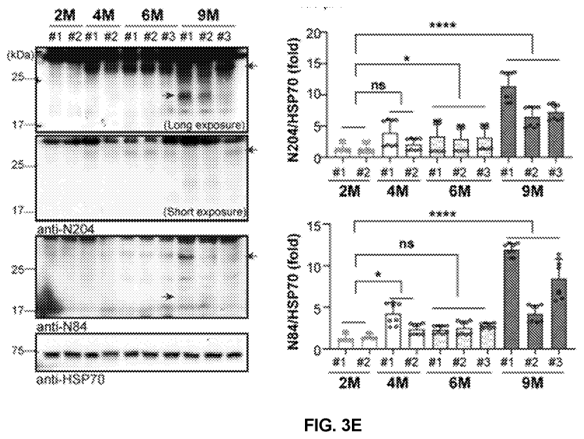

FIG. 3A is a schematic diagram illustrating the construction of EBP1 1-84 fragment- and EBP1 1-204 fragment-specific antibodies.

Peptides (KGIAFPTSISVNN, SEQ ID NO: 7, EBP1 71-84; and QHVIDGEKTIIQN, SEQ ID NO: 8, EBP1 191-204) with a purify of 95% or higher were synthesized on the basis of the sequences of 13 amino acids upstream of the asparagine 84 and asparagine 204 of the EBP1 protein, respectively. The peptides were injected into rabbits three times to give antiserum. Antibody production was conducted by Abclon.

Thereafter, an EBP1 fragment detectable by each antibody was confirmed by Western blotting, and then the antiserum was purified using IgG, and the reactivity of each antibody was measured by ELISA. EBP1 71-84 fragment- and EBP1 191-204 fragment-specific antibodies were utilized for in vitro and in vivo (brain tissue) Western blotting and immunostaining.

The results are shown in FIGS. 3B and 3D.

As shown in FIG. 3B, two fragments confirmed by the in vitro EBP1 protein cleavage experiment were detectable using EBP1 71-84 fragment- and EBP1 191-204 fragment-specific antibodies. It was also confirmed that the EBP1 fragments cleaved at either asparagine 84 or asparagine 204 (EBP1 1-84 or EBP1 1-204) could be detected by the above constructed antibodies.

As shown in FIG. 3C, as a result of comparing the detection of EBP1 1-84 and EBP1 1-204 fragments in the brain tissues of controls and Alzheimer's disease patients, the EBP1 1-84 and EBP1 1-204 fragments cleaved at asparagine 84 or 204 were observed only in the brain of Alzheimer′ disease patients.

As shown in FIG. 3D, as a result of the treatment with antibodies detecting endo-lysosomes (EEA1 and LAMP1) where amyloid beta cleavage occurs, and EBP1 71-84 fragment- and EBP1 191-204 fragment-specific antibodies in the brain tissues of controls and Alzheimer's disease patients, EEA1, LAMP1, EBP1 1-84, and EBP1 1-204 were detected together. That is, it was confirmed that the EBP1 1-84 and EBP1 1-204 fragments were cleaved in the endo-lysosomes by active AEP.

In addition, the present inventors performed Western blotting using brain tissues collected at different month ages in the Alzheimer animal model 5X-FAD mice. The results are shown FIG. 3E.

As shown in FIG. 3E, protein fragments cleaved by AEP in the brain tissues of 5X-FAD mice at 9 months of age were detected by anti-N84 and anti-N204 antibodies. The above results confirmed that EBP1 protein was cleaved by AEP in the brains of mice exhibiting aggravated Alzheimer's disease pathological phenotypes.

### Example 4: Confirmation of Neuronal Toxicity of EBP1 Protein Fragments

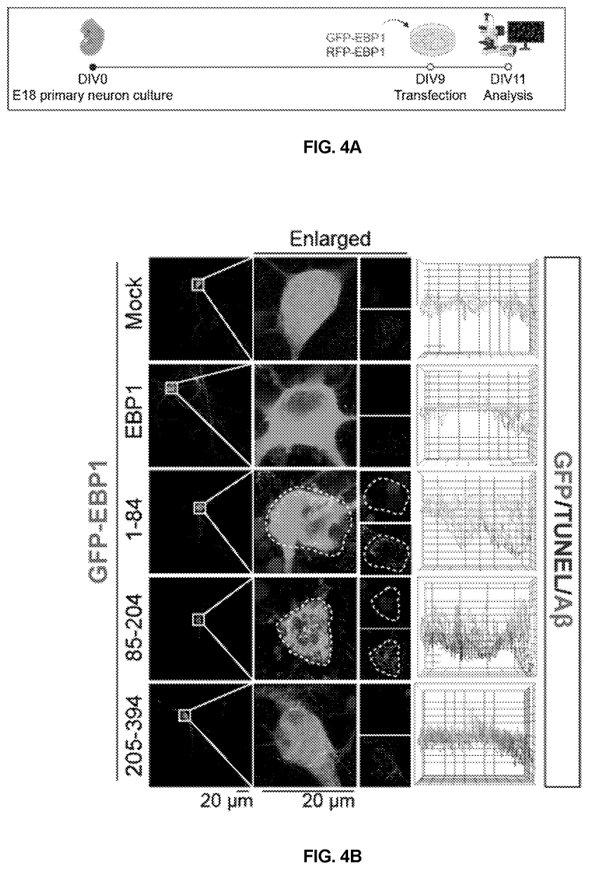

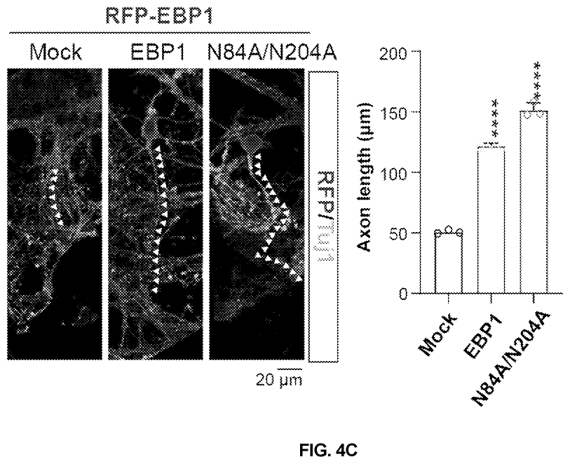

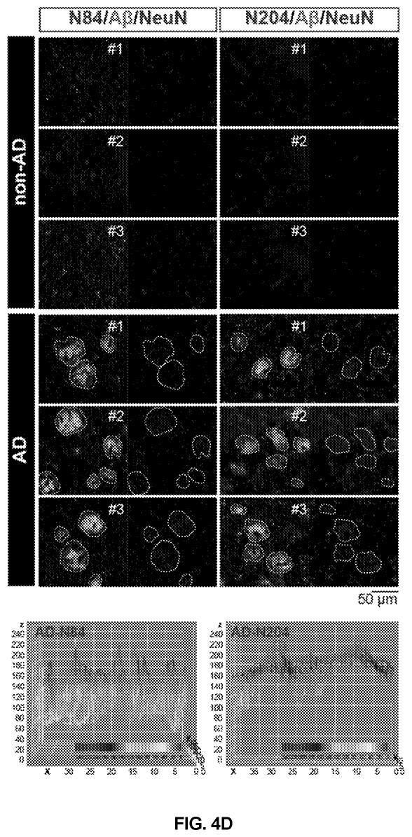

FIG. 4A is a schematic diagram of experimentation for investigating cytotoxicity by the EBP1 protein fragments EBP1 1-84 and EBP1 1-204.

TUNEL staining was performed to evaluate apoptosis in brain tissues of mice and primary hippocampal neurons overexpressing GFP-EBP1 plasmids. First, hippocampal neurons were transfected with GFP-EBP1 fragments, and the hippocampal neurons overexpressing the GFP-EBP1 fragments were fixed in 4% paraformaldehyde (PFA) and stained with anti-amyloid beta (anti-AP) and anti-NeuN antibodies. In such a procedure, in situ TUNEL analysis was performed to detect apoptosis. A Cell Death Detection kit (Roche, #12156792910) was used to perform staining according to the manufacturer's procedure.

As shown in FIG. 4B, the TUNEL histological staining results confirmed apoptosis in the primary hippocampal neurons overexpressing EBP1 1-84 and EBP1 85-204 corresponding to cleaved EBP1 fragments at asparagine 84 and/or asparagine 204 of the EBP1 protein.

As shown in FIG. 4C, as a result of overexpressing red fluorescent protein-tagged EBP1 and EBP1 N84A/N204A plasmids in primary neurons obtained from embryos of 5X-FAD mice corresponding to an Alzheimer's disease animal model, the EBP1 protein was not cleaved by AEP, restoring the axon length of the neurons

As shown in FIG. 4D, the EBP1 1-84 and EBP1 1-204 fragments were accumulated together with amyloid beta in the brain tissues of Alzheimer's disease patients.

### Example 5: Identification of the Ability of EBP1 to Regulate Gamma-Secretase Activity

To investigate the relationship between EBP1 protein and gamma-secretase activity, gamma-secretase activity was analyzed using luciferase with or without cleavage of EBP1 protein and with or without the administration of EBP1 protein.

Conditional knockout (CKO) mice with forebrain-specific deletion of the EBP1 gene were manufactured. The EBP1 gene is located on chromosome 10. To generate EBP1 conditional knockout mice (EBP1^{flox/flox} mice) using the Cre-loxP system, loxP was inserted upstream and downstream of exons 6-10 of the EBP1 gene on mouse chromosome 10. Forebrain-specific EBP1 conditional knockout (CKO) mice were generated by crossing EBP1^{flox/flox} mice with mice having CamkII-Cre promoter. When Cre recombinase is expressed in the CKO mice, the region between the loxP cassettes is excised, and EBP1 is inactivated in a cell type-specific manner in cells expressing CamkII, thereby resulting in forebrain-specific deletion of the EBP1 gene. All procedures for experimental animals were approved by the Institutional Animal Care and Use Committee (IACUC) of Sungkyunkwan University School of Medicine and were conducted according to IACUC guidelines.

Luciferase assays for measuring the gamma-secretase activity by the EBP1 1-84 and 85-204 fragments were performed using a dual luciferase assay kit (Promega WT, U.S., #E1980) according to the manufacturer's instructions.

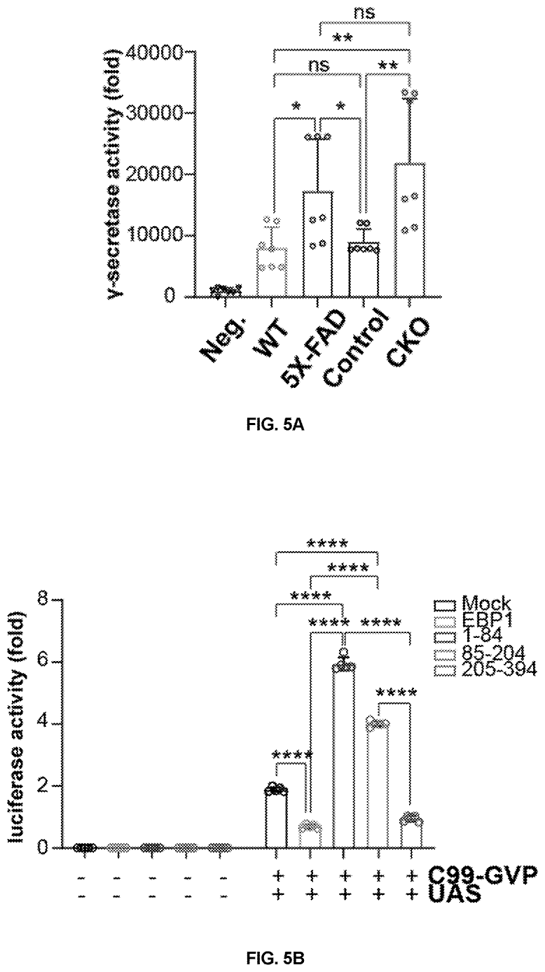

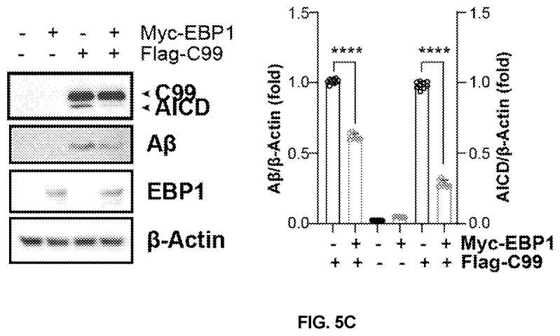

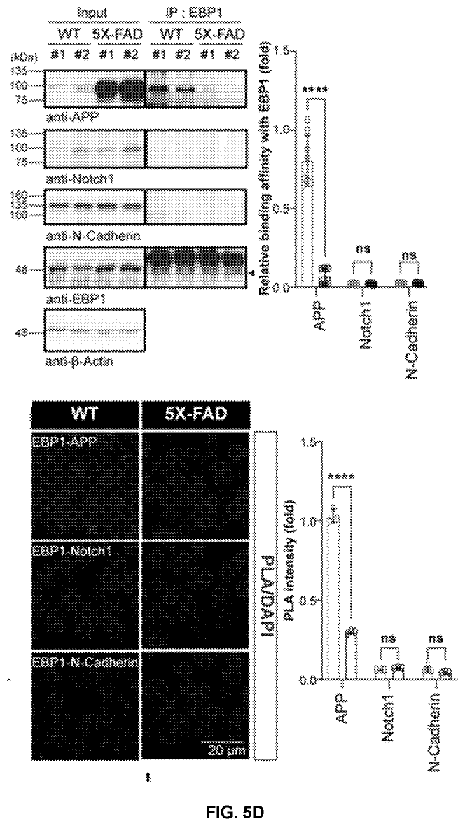

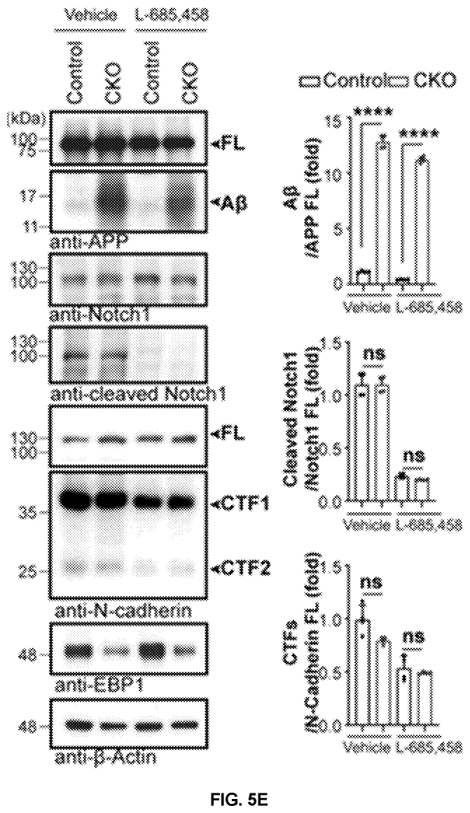

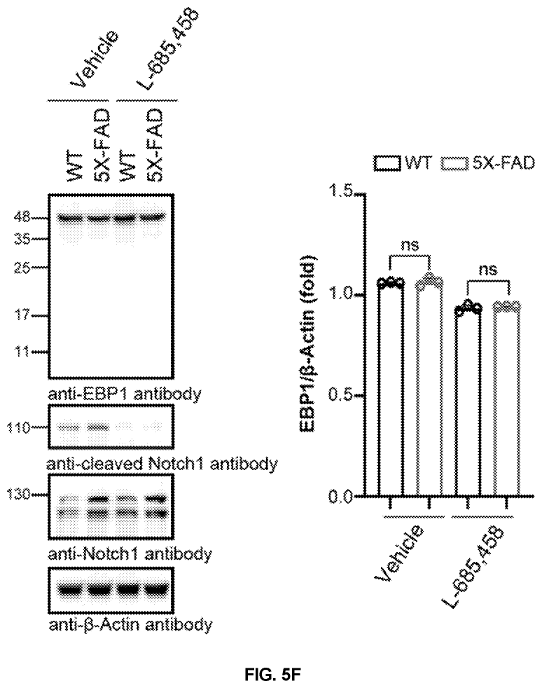

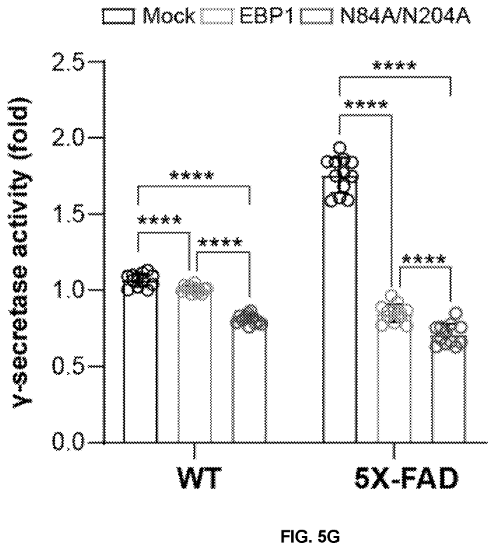

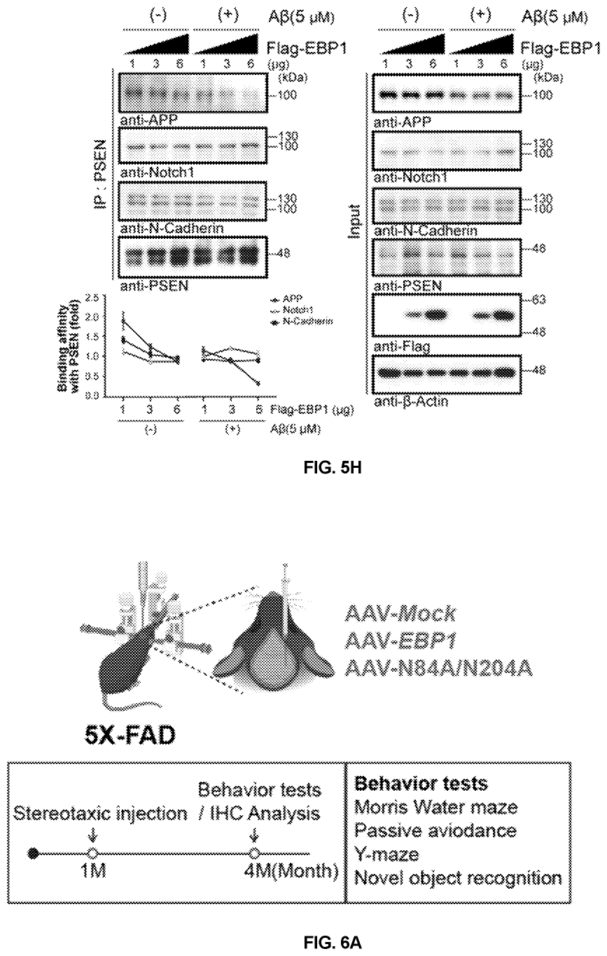

The results are shown FIG. 5.

As shown in FIG. 5A, the gamma-secretase activity in the brain of the forebrain-specific EBP1 conditional knockout (CKO) mice was increased to that in Alzheimer's disease animal model mice 5X-FAD.

As shown in FIG. 5B, the EBP1 1-84 and 85-204 fragments cleaved by AEP were confirmed to increase the gamma-secretase activity.

The cleavage of amyloid beta precursor protein (APP) by beta-secretase results in C99. The cleavage of C99 by gamma-secretase activated by EBP1 fragments results in AICD and amyloid beta production.

As shown in FIG. 5C, both AICD and amyloid beta were reduced due to EBP1 expression.

As shown in FIG. 5D, EBP1 was confirmed to specifically bind to APP among gamma-secretase substrates. In addition, a strong binding between EBP1 and APP was maintained in the brains of normal animals (WT), but the binding affinity was reduced in the brains of 5X-FAD. These results indicate that the gamma-secretase inhibitory effect of EBP1 was diminished.

As shown in FIG. 5E, EBP1 specifically regulated the activity of gamma-secretase on APP and did not affect the cleavage of representative substrates of gamma-secretase, such as Notch1 or N-Cadherin.

As shown in FIG. 5F, EBP1 was not cleaved by gamma-secretase.

As shown in FIG. 5G, the overexpression of EBP1 wild-type and N84A/N204A in the primary neurons of 5X-FAD mice reduced the gamma-secretase activity.

It was therefore confirmed that the overexpression of EBP1 protein or the EBP1 N84A/N204A mutant protein, which was mutated to be uncleavable by APP, reduced the activity of gamma-secretase on APP.

Furthermore, primary neurons were cultured, treated with amyloid beta, and then overexpressed with Flag-EBP1 plasmid (1, 3, and 6 μg each). After cell lysis, the binding degrees between PSEN and other substrates were compared through immunoprecipitation. The results are shown in FIG. 5H.

As shown in FIG. 5H, in the primary neurons treated with amyloid beta, the binding between APP and PSEN with gamma-secretase activity decreased as the EBP1 expression increased. From these experimental results, the amyloid beta treatment means the Alzheimer's disease phenotype, and the above results indicate that as the EBP1 expression increased in the Alzheimer's diseases, the binding between PSEN and APP decreased. It was ultimately confirmed that, the production of amyloid beta decreased with an increase in EBP1. In addition, it was confirmed that the bindings of PSEN to Notch1 and N-Cadherin were not affected by EBP1.

### Example 6: Analysis of Effect of EBP1 Wild-Type or N84A/N204A Overexpression on Amyloid Beta

### 6-1. EBP1 Gene Cloning and AAV Viral Vector Construction

### 6-1-1. EBP1 Cloning

A pair of nucleotide primers (EBP1-F: 5′-gaattcatgtcgggcgagga-cgag-3′ (SEQ ID NO: 9) and EBP1-R: 5′-ctcgagtcagtccccagcttcattttct-3′ (SEQ ID NO: 10)) corresponding to an open reading frame (ORF) based on the Homo sapiens cDNA [PA2G4, GenBank accession number NM_006191.3] was prepared from the NCBI database. The full-length open reading frame (ORF) of EBP1 was obtained by performing PCR and cloning based on cDNA synthesized from mRNA extracted from HEK-293T cells. A pair of primers capable of inducing mutations of asparagine 84 and 204 to alanine was prepared and utilized to perform PCR on the basis of wild-type EBP1, thereby constructing EBP1 N84A/204A.

### 6-1-2. EBP1 Adeno-Associated Virus (AAV) Production

To expression EBP1 in AAV, the EBP1 open reading frame obtained in the Preparation Example 1-1 was subcloned into the pAAV-CMV vector, an AAV expression vector. The pAAV-CMV-EBP1, Helper, and Re-cap vectors were co-expressed to produce viruses, and AAV production was commissioned to the Virus Center at the Korea Institute of Science and Technology (KIST).

### 6-2. Effects of EBP1 Wild-Type or N84A/N204A Overexpression on Amyloid Beta

### 6-2-1. Animal Models

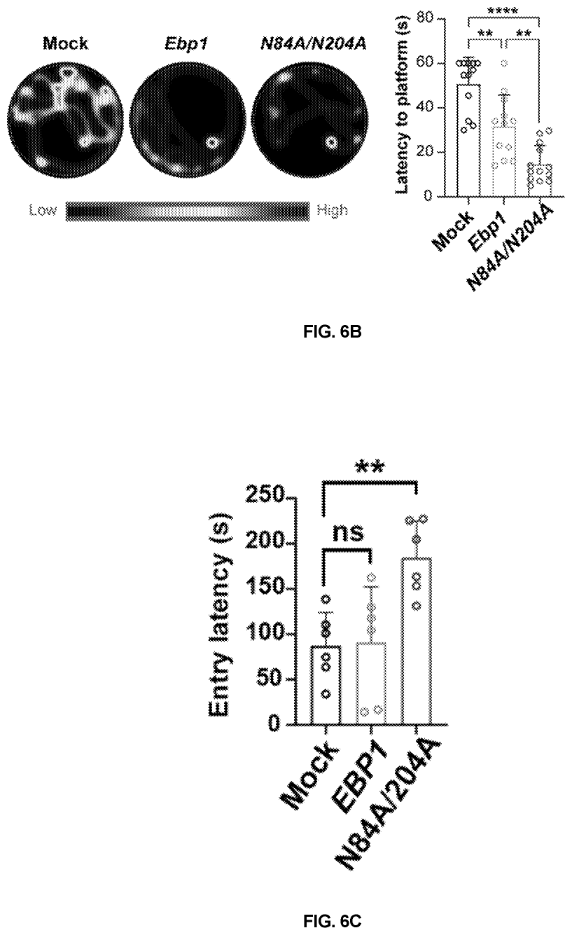

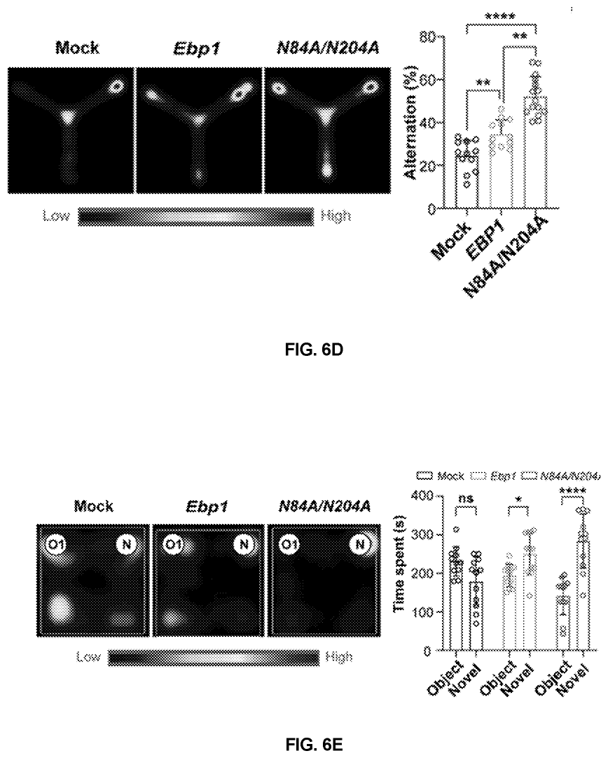

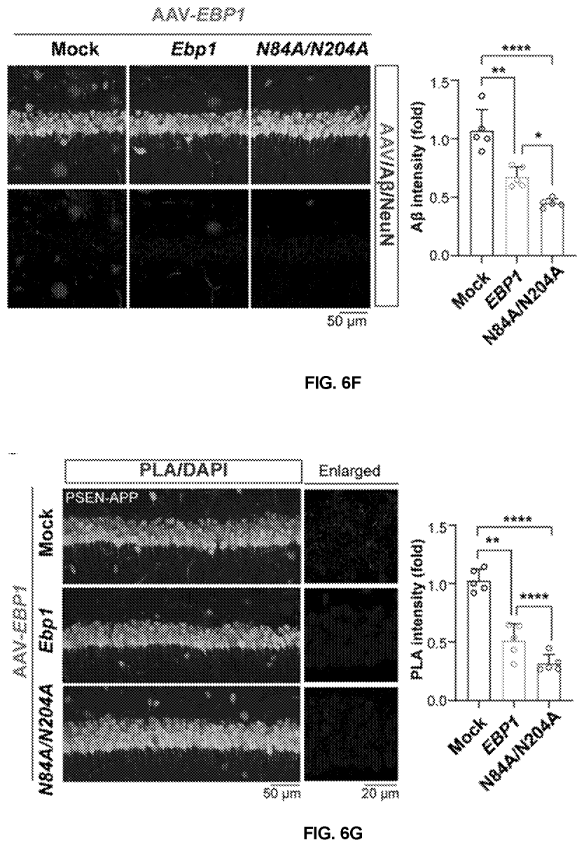

FIG. 6A is a schematic diagram of process of behavioral tests and immunohistochemistry analysis four months after the injection of AAV-EBP1 and AAV-N84A/204A into the hippocampus of 1-month-old 5X-FAD mice. 5X-FAD mice, widely known as Alzheimer's disease animal model, were used.

AAV-Mock, AAV-EBP1, and AAV-EBP1 N84A/N204A were injected into the CA1 region of the hippocampus in 1-month-old 5X-FAD mice, prior to the accumulation of amyloid beta. After the mouse head was fixed in a stereotaxic frame and then shaved, a 10-μl Hamilton syringe was inserted into the hippocampus from the bregma and then 1 μl of viruses was injected. The virus titers of AAV-Mock, AAV-EBP1, and AAV-EBP1 N84A/N204A were 8.63×10¹² GC/ml, 2.41×10¹² GC/ml, and 2.84×10¹² GC/ml, respectively, which were different from each other.

To evaluate the cognitive function of the animal models constructed by the above method, representative behavioral tests were performed. Specifically, Morris water maze test, passive avoidance test, Y-maze test, and novel object recognition test were performed as cognitive function evaluation experiments.

Randomly assigned age- and sex-matched mice were used. 5X-FAD mice injected with AAV-Mock (Control), AAV-EBP1 or AAV-N84A/N204A (n=12, AAV-Mock and AAV-EBP1/n=13, AAV-N84A/N204A) were used. In the habituation phase before the trials, mice were placed in empty cages to adapt to the environment for 10 min. The movement of each mouse was recorded and analyzed using the video-tracking software EthoVision XT14 (Noldus, Netherlands).

### 6-2-2. Morris Water Maze

Before the test, the Morris water maze consisted of a pool (150 cm in diameter with 60 cm in height of walls) filled with water set to 22° C. The pool was divided into four quarters, one of which included a transparent platform (9 cm in diameter, 25 cm in height). The test consisted of a 3-day training trial and a 1-day probe trial. Mice were subjected to training trials three times a day for 60 s at one time, with 1-h intervals between the experimental sessions. The escape latency was recorded three times a day during training sessions. The test phase was carried out 24 h after the last test trial. All the other conditions were kept the same as the test trial, except for the removal of the hidden platform.

As confirmed by the results shown in FIG. 6B, the latency to find the target was significantly decreased in the 5X-FAD mice overexpressing AAV-EBP1 and/or AAV-N84A/N204A.

### 6-2-3. Passive Avoidance Test

The test apparatus consisted of a transparent room and a dark room connected, with a barrier wall therebetween. The bottom of the apparatus had metal bars to conduct electricity. In the training session, mice were initially placed in the transparent room. After exploration, the mouse entered the dark room, the door was then closed and an electric shock of 0.4-mA intensity was applied for 2 s. In the test session, mice subjected to electric shock were placed in the transparent room, and the time until they entered the dark room was measured. The cutoff time of the test session was up to 300 s. The experiment proceeded in a random order, and the experimenter was blinded such that they could not know which experiment was in progress and which mouse was a subject of the experiment.

As confirmed by the results shown in FIG. 6C, the overexpression of EBP1 N84A/N204A led to the highest level of learning and memory in the mice. Therefore, the passive avoidance test result confirmed that the overexpression of EBP1 N84A/N204A enhanced memory performance.

### 6-2-4. Y-Maze Test

The test was performed using the tendency to move to the opposite arm in a symmetrical Y-maze. Each mouse was placed along the side wall of one arm of the maze and allowed to explore the maze for 10 min.

As confirmed by the Y-maze test results shown in FIG. 6D, 5X-FAD mice overexpressing AAV-EBP1 and AAV-N84A/N204A exhibited an improved ability to explore a novel environment. Therefore, the Y-maze test results confirmed that the overexpression of EBP1 or EBP1 N84A/N204A led to an enhancement in cognitive function.

### 6-2-5. Novel Object Recognition Test

The novel object recognition test consisted of three phases: a habituation phase; a familiarization phase; and a test phase. The habituation phase is identical to other behavior tests. In the familiarization phase, mice were placed in an open cage with two identical objects. In the test phase 24 h after the familiarization phase, one of the two objects was replaced with a novel object, and the time during which the mouse's nose touched the object or the mouse was oriented toward the object within 2 cm was recorded for 10 min. Preferential recognition indicates a behavior where a mouse distinguishes and prefers a novel object from an existing object or spends more time, and this behavior is interpreted as being associated with memory and learning performance in the cognitive functions.

As confirmed by the results shown in FIG. 6E, the time spent to recognize the novel object was significantly increased in the EBP1- and EBP1 N84A/204A-injected mice, indicating the mitigation of impaired preferential recognition of the novel object.

### 6-2-6. Mouse Brain Tissue Staining

To determine whether amyloid beta accumulation and new amyloid beta production were reduced in the brain tissues of EBP1- and N84A/N204A-overexpressing mice, the brain tissues of the mice were stained and observed.

The CA1 regions of the EBP1- and N84A/N204A-overexpressed 5X-FAD mice were stained. The results are shown in FIGS. 6F and 6G.

As shown in FIG. 6F, the overexpression of EBP1 and N84A/N204 reduced amyloid beta accumulations. As shown in FIG. 6G, the overexpression of EBP1 and N84A/N204 reduced the binding between APP and PSEN, leading to a reduction in amyloid beta production.

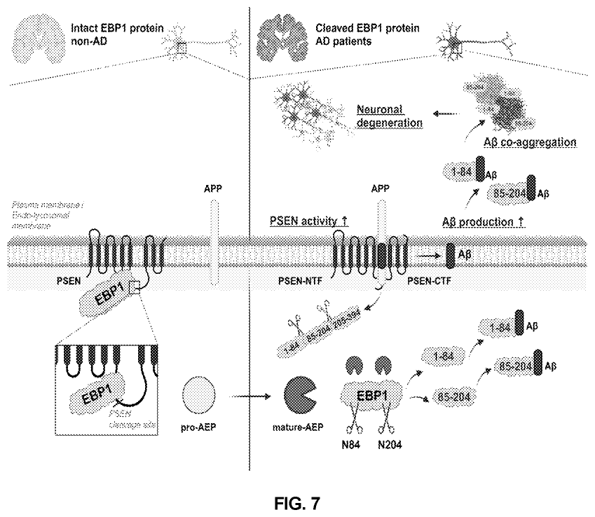

Taken together, as shown in the schematic diagram of FIG. 7, the composition of the present disclosure targets the mechanism where the gamma-secretase is deregulated due to AEP-mediated EBP1 cleavage, causing neurodegeneration and accelerating amyloid beta production. Thus the composition of the present disclosure can be utilized as a pharmaceutical composition for the prevention or early treatment for Alzheimer's disease.

What is claimed is:

1. A pharmaceutical composition for preventing or treating a brain disease, the pharmaceutical composition comprising as an active ingredient an ErbB3-binding protein 1 (EBP1) protein including an amino acid sequence set forth in any one of SEQ ID NOs: 1 to 3 or a nucleic acid molecule including a polynucleotide sequence encoding the EBP1 protein.

2. The pharmaceutical composition of claim 1, wherein the polynucleotide sequence includes a nucleotide sequence set forth in any one of SEQ ID NOs: 4 to 6.

3. The pharmaceutical composition of claim 1, wherein the nucleic acid molecule is a naked DNA, or is contained in a gene delivery carrier.

4. The pharmaceutical composition of claim 3, wherein the gene delivery carrier is a vector.

5. The pharmaceutical composition of claim 4, wherein the vector is a plasmid vector, a cosmid vector, or a viral vector.

6. The pharmaceutical composition of claim 1, wherein the brain disease is Alzheimer's disease.

7. A method for preventing or treating a brain disease, the method comprising administering a pharmaceutical composition to a subject, the pharmaceutical composition comprising as an active ingredient an ErbB3-binding protein 1 (EBP1) protein including an amino acid sequence set forth in any one of SEQ ID NOs: 1 to 3 or a nucleic acid molecule including a polynucleotide sequence encoding the EBP1 protein.

8. The method of claim 7, wherein the polynucleotide sequence includes a nucleotide sequence set forth in any one of SEQ ID NOs: 4 to 6.

9. The method of claim 7, wherein the nucleic acid molecule is a naked DNA, or is contained in a gene delivery carrier.

10. The method of claim 9, wherein the gene delivery carrier is a vector.

11. The method of claim 10, wherein the vector is a plasmid vector, a cosmid vector, or a viral vector.

12. The method of claim 7, wherein the brain disease is Alzheimer's disease.


SEQUENCE LISTING (10 sequences)

SEQ ID NO: 1 - protein, 394 aa, synthetic construct
MSGEDEQQEQTIAEDLVVTKYKMGGDIANRVLRSLVEASSSGVSVLSLCEKGDAMIMEETGKIFKKEKEMKKGIAFPTSISVNACVCHFSPLKSDQDYILKEGDLVKIDLGVHVDGFIANVAHTFVVDVAQGTQVTGRKADVIKAAHLCAEAALRLVKPGNQNTQVTEAWNKVAHSFNCTPIEGMLSHQLKQHVIDGEKTIIQNPTDQQKKDHEKAEFEVHEVYAVDVLVSSGEGKAKDAGQRTTIYKRDPSKQYGLKMKTSRAFFSEVERRFDAMPFTLRAFEDEKKARMGVVECAKHELLQPFNVLYEKEGEFVAQFKFTVLLMPNGPMRITSGPFEPDLYKSEMEVQDAELKALLQSSASRKTQKKKKKKASKTAENATSGETLEENEAGD

SEQ ID NO: 2 - protein, 394 aa, synthetic construct
MSGEDEQQEQTIAEDLVVTKYKMGGDIANRVLRSLVEASSSGVSVLSLCEKGDAMIMEETGKIFKKEKEMKKGIAFPTSISVNNCVCHFSPLKSDQDYILKEGDLVKIDLGVHVDGFIANVAHTFVVDVAQGTQVTGRKADVIKAAHLCAEAALRLVKPGNQNTQVTEAWNKVAHSFNCTPIEGMLSHQLKQHVIDGEKTIIQAPTDQQKKDHEKAEFEVHEVYAVDVLVSSGEGKAKDAGQRTTIYKRDPSKQYGLKMKTSRAFFSEVERRFDAMPFTLRAFEDEKKARMGVVECAKHELLQPFNVLYEKEGEFVAQFKFTVLLMPNGPMRITSGPFEPDLYKSEMEVQDAELKALLQSSASRKTQKKKKKKASKTAENATSGETLEENEAGD

SEQ ID NO: 3 - protein, 394 aa, synthetic construct
MSGEDEQQEQTIAEDLVVTKYKMGGDIANRVLRSLVEASSSGVSVLSLCEKGDAMIMEETGKIFKKEKEMKKGIAFPTSISVNACVCHFSPLKSDQDYILKEGDLVKIDLGVHVDGFIANVAHTFVVDVAQGTQVTGRKADVIKAAHLCAEAALRLVKPGNQNTQVTEAWNKVAHSFNCTPIEGMLSHQLKQHVIDGEKTIIQAPTDQQKKDHEKAEFEVHEVYAVDVLVSSGEGKAKDAGQRTTIYKRDPSKQYGLKMKTSRAFFSEVERRFDAMPFTLRAFEDEKKARMGVVECAKHELLQPFNVLYEKEGEFVAQFKFTVLLMPNGPMRITSGPFEPDLYKSEMEVQDAELKALLQSSASRKTQKKKKKKASKTAENATSGETLEENEAGD

SEQ ID NO: 4 - DNA, 1185 bp, synthetic construct
  source 1..1185: mol_type = other DNA
atgtcgggcgaggacgagcaacaggagcaaactatcgctgaggacctggtcgtgaccaagtataagatggggggcgacatcgccaacagggtacttcggtccttggtggaagcatctagctcaggtgtgtcggtactgagcctgtgtgagaaaggtgatgccatgattatggaagaaacagggaaaatcttcaagaaagaaaaggaaatgaagaaaggtattgcttttcccaccagcatttcggtaaatgcctgtgtatgtcacttctcccctttgaagagcgaccaggattatattctcaaggaaggtgacttggtaaaaattgaccttggggtccatgtggatggcttcatcgctaatgtagctcacacttttgtggttgatgtagctcaggggacccaagtaacagggaggaaagcagatgttattaaggcagctcacctttgtgctgaagctgccctacgcctggtcaaacctggaaatcagaacacacaagtgacagaagcctggaacaaagttgcccactcatttaactgcacgccaatagaaggtatgctgtcacaccagttgaagcagcatgtcatcgatggagaaaaaaccattatccagaatcccacagaccagcagaagaaggaccatgaaaaagctgaatttgaggtacatgaagtatatgctgtggatgttctcgtcagctcaggagagggcaaggccaaggatgcaggacagagaaccactatttacaaacgagacccctctaaacagtatggactgaaaatgaaaacttcacgtgccttcttcagtgaggtggaaaggcgttttgatgccatgccgtttactttaagagcatttgaagatgagaagaaggctcggatgggtgtggtggagtgcgccaaacatgaactgctgcaaccatttaatgttctctatgagaaggagggtgaatttgttgcccagtttaaatttacagttctgctcatgcccaatggccccatgcggataaccagtggtcccttcgagcctgacctctacaagtctgagatggaggtccaggatgcagagctaaaggccctcctccagagttctgcaagtcgaaaaacccagaaaaagaaaaaaaagaaggcctccaagactgcagagaatgccaccagtggggaaacattagaagaaaatgaagctggggactga

SEQ ID NO: 5 - DNA, 1185 bp, synthetic construct
  source 1..1185: mol_type = other DNA
atgtcgggcgaggacgagcaacaggagcaaactatcgctgaggacctggtcgtgaccaagtataagatggggggcgacatcgccaacagggtacttcggtccttggtggaagcatctagctcaggtgtgtcggtactgagcctgtgtgagaaaggtgatgccatgattatggaagaaacagggaaaatcttcaagaaagaaaaggaaatgaagaaaggtattgcttttcccaccagcatttcggtaaataactgtgtatgtcacttctcccctttgaagagcgaccaggattatattctcaaggaaggtgacttggtaaaaattgaccttggggtccatgtggatggcttcatcgctaatgtagctcacacttttgtggttgatgtagctcaggggacccaagtaacagggaggaaagcagatgttattaaggcagctcacctttgtgctgaagctgccctacgcctggtcaaacctggaaatcagaacacacaagtgacagaagcctggaacaaagttgcccactcatttaactgcacgccaatagaaggtatgctgtcacaccagttgaagcagcatgtcatcgatggagaaaaaaccattatccaggctcccacagaccagcagaagaaggaccatgaaaaagctgaatttgaggtacatgaagtatatgctgtggatgttctcgtcagctcaggagagggcaaggccaaggatgcaggacagagaaccactatttacaaacgagacccctctaaacagtatggactgaaaatgaaaacttcacgtgccttcttcagtgaggtggaaaggcgttttgatgccatgccgtttactttaagagcatttgaagatgagaagaaggctcggatgggtgtggtggagtgcgccaaacatgaactgctgcaaccatttaatgttctctatgagaaggagggtgaatttgttgcccagtttaaatttacagttctgctcatgcccaatggccccatgcggataaccagtggtcccttcgagcctgacctctacaagtctgagatggaggtccaggatgcagagctaaaggccctcctccagagttctgcaagtcgaaaaacccagaaaaagaaaaaaaagaaggcctccaagactgcagagaatgccaccagtggggaaacattagaagaaaatgaagctggggactga

SEQ ID NO: 6 - DNA, 1185 bp, synthetic construct
  source 1..1185: mol_type = other DNA
atgtcgggcgaggacgagcaacaggagcaaactatcgctgaggacctggtcgtgaccaagtataagatggggggcgacatcgccaacagggtacttcggtccttggtggaagcatctagctcaggtgtgtcggtactgagcctgtgtgagaaaggtgatgccatgattatggaagaaacagggaaaatcttcaagaaagaaaaggaaatgaagaaaggtattgcttttcccaccagcatttcggtaaatgcctgtgtatgtcacttctcccctttgaagagcgaccaggattatattctcaaggaaggtgacttggtaaaaattgaccttggggtccatgtggatggcttcatcgctaatgtagctcacacttttgtggttgatgtagctcaggggacccaagtaacagggaggaaagcagatgttattaaggcagctcacctttgtgctgaagctgccctacgcctggtcaaacctggaaatcagaacacacaagtgacagaagcctggaacaaagttgcccactcatttaactgcacgccaatagaaggtatgctgtcacaccagttgaagcagcatgtcatcgatggagaaaaaaccattatccaggctcccacagaccagcagaagaaggaccatgaaaaagctgaatttgaggtacatgaagtatatgctgtggatgttctcgtcagctcaggagagggcaaggccaaggatgcaggacagagaaccactatttacaaacgagacccctctaaacagtatggactgaaaatgaaaacttcacgtgccttcttcagtgaggtggaaaggcgttttgatgccatgccgtttactttaagagcatttgaagatgagaagaaggctcggatgggtgtggtggagtgcgccaaacatgaactgctgcaaccatttaatgttctctatgagaaggagggtgaatttgttgcccagtttaaatttacagttctgctcatgcccaatggccccatgcggataaccagtggtcccttcgagcctgacctctacaagtctgagatggaggtccaggatgcagagctaaaggccctcctccagagttctgcaagtcgaaaaacccagaaaaagaaaaaaaagaaggcctccaagactgcagagaatgccaccagtggggaaacattagaagaaaatgaagctggggactga

SEQ ID NO: 7 - protein, 13 aa, synthetic construct
KGIAFPTSISVNN

SEQ ID NO: 8 - protein, 13 aa, synthetic construct
QHVIDGEKTIIQN

SEQ ID NO: 9 - DNA, 24 bp, synthetic construct
  source 1..24: mol_type = other DNA
gaattcatgtcgggcgaggacgag

SEQ ID NO: 10 - DNA, 28 bp, synthetic construct
  source 1..28: mol_type = other DNA
ctcgagtcagtccccagcttcattttct